(19) United States
(12) Patent Application Publication
(10) Pub. No.: US 2018/0116691 A1
(43) Pub. Date: May 3, 2018

(54) MULTI-LAYER SEAL WITH OVERLAPPING OPENINGS

(71) Applicant: Intuitive Surgical Operations, Inc., Sunnyvale, CA (US)

(72) Inventor: Robert C. Reid, Fairfield, CT (US)

(21) Appl. No.: 15/794,911

(22) Filed: Oct. 26, 2017

Related U.S. Application Data
(60) Provisional application No. 62/413,818, filed on Oct. 27, 2016.

(51) Int. Cl. A61B 17/34; A61B 34/35; A61M 13/00; A61B 17/00

(52) U.S. Cl. CPC A61B 17/3417; A61B 34/35; A61M 13/003; A61B 2034/302; A61B 2017/3419; A61B 2017/3429; A61B 17/00234

(57) ABSTRACT

A seal for sealing against a surgical instrument shaft includes a plurality of seal layers and a plurality of overlapping seal openings formed in the seal layers. The plurality of seal openings together can create an effective seal opening that is smaller than any of the individual overlapping seal openings. The effective seal opening is sized and shaped to seal against an instrument shaft when an instrument shaft is inserted through the effective seal opening.

### CLAIM OF PRIORITY

This application claims the benefit of priority under 35 U.S.C. § 119(e) to U.S. Provisional Patent Application Ser. No. 62/413,818, filed on Oct. 27, 2016, which is incorporated by reference herein in its entirety.

### TECHNICAL FIELD

This document relates generally to medical devices, and more particularly, to systems, devices and methods for sealing an opening in a body during a surgical procedure.

### BACKGROUND

Certain surgical procedures, such as minimally-invasive or laparoscopic surgery, can involve delivery of an insufflation of a gas into the body. For example, in a laparoscopic procedure, an insufflation gas can be delivered to the peritoneal cavity of a patient to distend the abdomen and improve visual and physical access to internal organs in the abdomen. Distension of the patient's abdomen can provide sufficient operating space enable adequate visualization of the structures and manipulation of instruments inside a patient.

It is important to maintain a sealed system to maintain insufflation during a surgical procedure. For example, the interface between surgical equipment and an access orifice in the patient's body must be sealed to avoid or reduce leakage of insufflation gas so that insufflation can be maintained.

In a less-invasive surgical procedure, such as a laparoscopic procedure, one or more cannulas are typically used to a deliver surgical tools into a body cavity. A cannula seal can be used to provide a seal between the cannula an outer surface of an instrument shaft that is connected to surgical tool to avoid or reduce leakage of insufflation gas through the cannula during the procedure. The cannula seal is a critical component for surgery in the abdomen, because without it there is no insufflation, and without insufflation surgery cannot be effectively carried out.

### SUMMARY

An example seal for sealing against a surgical instrument shaft can include a first seal layer having a first seal opening and a second seal layer having a second seal opening. The second seal opening can overlap with the first seal opening. An effective seal opening can be defined at least in part by the first seal layer and the second seal layer at a region where the first seal opening overlaps with the second seal opening. The effective seal opening can be smaller than the first seal opening and the second seal opening. The effective seal opening can be sized and shaped to seal against an instrument shaft when an instrument shaft is inserted through the effective seal opening.

In some examples, when an instrument shaft that is larger than the effective seal opening is inserted through the effective seal opening, the first seal opening or the second seal opening deforms to accommodate the instrument shaft.

In some examples, the first seal opening has a first center, and the second seal opening has a second center, and the second center is offset from the first center.

In some examples, the seal includes a third seal layer that has a third seal opening, and the third seal opening has a third center that is offset from the first center and the second center. In some examples, the seal also includes a fourth seal layer having a fourth seal opening, and the fourth seal opening has a fourth center that is offset from the first center, the second center, and the third center.

In some examples, the first seal opening and the second seal opening both have a circular shape.

In some examples, the first seal opening has an elliptical, ovular, or oblong shape.

In some examples, the first seal opening is a different size or different shape than the second seal opening.

In some examples, the first seal layer has a first thickness, and moving the instrument shaft off the axis a first distance creates less distortion in the effective opening than would occur in a comparable single-layer seal formed of a single layer having a comparable thickness that is equal to the first thickness and a comparable opening that is the same size and the effective opening.

In some examples, the seal includes a first seal component that includes a first end wall that includes the first seal layer and the first seal opening, and a first side wall connected to the first end wall, the first side wall extending around and defining a first interior chamber, and a second seal component that includes a second end wall that includes the second seal layer and the second seal opening, and a second side wall connected to the second end wall, the second side wall extending around and defining a second interior chamber, the second seal component sized and shaped to fit inside the first interior chamber of the first seal component. In some examples, the seal further includes a third seal component that includes a third end wall and a third side wall connected to the third end wall, the third side wall extending around a third interior chamber, the third seal component sized and shaped to fit inside the second interior chamber of the second seal component, the third end wall including a third seal layer and a third seal opening. In some examples, the seal further includes a fourth seal component that includes a fourth end wall and a fourth side wall connected to the fourth end wall, the fourth side wall extending around a fourth interior chamber, the fourth seal component sized and shaped to fit inside the third interior chamber of the third seal component, the fourth end wall including a fourth seal layer and a fourth seal opening in the fourth seal layer, the first seal layer, the second seal layer, the third seal layer, and the fourth seal layer defining the effective seal opening at a region where the first seal opening, the second seal opening, the third seal opening, and the fourth seal opening overlap.

An example multi-layer instrument seal can include a first seal component having a first seal wall and a first opening in the first seal wall, and a first side wall connected to and extending around the first seal wall, the first side wall defining a first interior chamber, and a second seal component having a second seal wall and a second opening in the second seal wall, and a second side wall connected to and extending around the second seal wall, the second side wall defining a second interior chamber, the second seal component can be sized and shaped to fit inside the first interior chamber of the first seal component, and a third seal component having a third seal wall and a third opening in the third seal wall, and a third side wall connected to and extending around the third seal wall, the third side wall defining a third interior chamber, the third seal component can be sized and shaped to fit inside the second interior chamber of the second seal component. The first opening, the second opening, and third opening can overlap with each other. An effective opening can be defined inside a region that intersects with the first opening, the second opening, and the third opening. The multi-layer instrument seal can be sized and shaped to seal against an instrument shaft when the instrument shaft extends through first opening, the second opening, and the third opening.

In some example, the multi-layer instrument seal can further include a fourth seal component having a fourth seal wall and a fourth opening in the fourth seal wall, and a fourth side wall connected to and extending around the fourth seal wall, the fourth side wall defining a fourth interior chamber, the fourth seal component can be sized and shaped to fit inside the third interior chamber of the third seal component. The effective opening can be defined by a region that intersects with the first opening, the second opening, the third opening, and the fourth opening. In some examples, the first opening, the second opening, and the third opening are equally spaced from each other. In some examples, the first opening, the second opening, and the third opening are the same size.

In some examples, an instrument shaft that has a cross-section that is larger than the effective opening can be inserted through the effective opening, and the seal applies normal forces against the instrument shaft that are smaller than the forces that would be created if the seal was a single layer of thickness and had an opening that is coextensive with the effective opening.

In some examples, the multi-layer instrument seal has a proximal opening. The proximal opening and the effective opening can define an axis extending from the proximal opening to the effective opening. The instrument shaft can be insertable through the proximal opening and the effective opening along the axis. In a first state the instrument shaft is in a first position aligned with the axis. In a second state the instrument shaft can be at a second position offset from the axis, in the second state one or more of the first seal wall, the second seal wall, and the third seal wall are stretched to deform one or more of the first opening, the second opening, or the third opening. In some examples, by portions of first seal component, second seal component, and third seal component that define the effective opening seal against the instrument shaft in both the first state and the second state.

In some examples, in a third state the instrument shaft is offset from the axis to a third position that is different from the second position, and when instrument shaft is moved from the second position to the third position, one or more of the first seal wall, the second seal wall, and the third seal wall are stretched, and one or more of the first seal wall, the second seal wall, and the third seal wall are relaxed, so that the by portions of first seal component, second seal component, and third seal component that define the effective opening seal against the instrument shaft in the third state.

An example method can include receiving an instrument shaft along an axis through an effective opening in a multi-layer seal that seals against the instrument shaft, at least a portion of the effective opening formed by a first seal layer having a first opening and a second seal layer having a second opening, the second opening overlapping and not coextensive with the first opening. When the instrument shaft is moved to a first position off the axis, one or more of the first seal layer and the second seal layer can be stretched to move the effective opening toward the first position of the instrument shaft in some examples, when the instrument shaft is moved from the first position to a second position off the axis, the first seal layer can be relaxed and the second seal layer can be stretched to adjust the position of the effective opening to match the second position of the instrument shaft.

In some examples, the first seal layer has a first thickness, and moving the instrument shaft off the axis a first distance creates less distortion in the effective opening than would occur in a comparable single-layer seal formed of a single layer having a comparable thickness that is equal to the first thickness and a comparable opening that is the same size and the effective opening.

Each of these non-limiting examples can stand on its own, or can be combined in various permutations or combinations with one or more of the other examples.

This Summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is an illustration of an example instrument system for use in teleoperated minimally invasive surgery.

FIG. 1B is an illustration of an example physician console for use in teleoperated minimally invasive surgery.

FIG. 1C is an illustration of an example control cart for use in teleoperated minimally invasive surgery.

FIG. 2A is a perspective view of an example expandable seal assembly with an instrument shaft inserted through the assembly.

FIG. 2B is an illustration of an expandable seal assembly utilized in a minimally-invasive surgical procedure.

FIG. 3A is a perspective illustration of an example seal component that has seal layers with overlapping openings.

FIG. 3B is an enlarged view of a portion of the seal component of FIG. 3A, showing the overlapping openings in the seal layers.

FIG. 3C is a top plan view of the seal component of FIG. 3A.

FIG. 3D is a top plan view of another example seal component showing an alternate arrangement of overlapping openings.

FIG. 4A is a top view of a seal assembly incorporating the seal component of FIG. 3A.

FIG. 4B is a side cross-sectional view of the seal assembly of FIG. 4A.

FIG. 4C is an enlarged view of a portion of the seal assembly of FIG. 4A that shows the layers that define the profile of the effective opening of the seal component.

FIG. 4D is a top view of a portion of a seal assembly that includes an example seal component that has elliptical openings in layers of a seal wall.

FIG. 5A is an exploded assembly view of four seal sub-components.

FIG. 5B is a perspective view of a first type of seal sub-component.

FIG. 5C is a perspective view of a second type of seal sub-component.

FIG. 6A is a top plan view of a portion of a seal component illustrating an example arrangement of four circular openings.

FIG. 6B is a top plan view of the portion of the seal component of FIG. 6A illustrating a shaft inserted through the effective openings.

FIG. 6C is a top plan view of a portion of a seal component illustrating an example arrangement of three circular openings.

FIG. 6D is a top plan view of a portion of a seal component illustrating another example arrangement of three circular openings.

FIG. 6E is a top plan view of a portion of a seal component illustrating an example arrangement of five circular openings.

FIG. 6F is a top plan view of a portion of a seal component illustrating an example arrangement of six circular openings.

FIG. 6G is a top plan view of a portion of a seal component illustrating an example arrangement of four elliptical openings.

FIG. 6H is a top plan view of a portion of a seal component illustrating another example arrangement of four elliptical openings.

FIG. 6I is a top plan view of a portion of a seal component illustrating an example arrangement of three elliptical openings.

FIG. 6J is a top plan view of a portion of a seal component illustrating another example arrangement of three elliptical openings.

FIG. 6K is a top plan view of a portion of a seal component in which one opening is a circle and two openings are ellipses.

### DETAILED DESCRIPTION

In some surgeries, it is desirable to exchange tools during the surgical procedure. For example, different visualization tools or surgical tools can be required at different points during a procedure, or based upon events or discoveries during the procedure. Exchanging a tool during a procedure can present a problem, however, as seals are frequently designed to accommodate a particular shaft size. A mismatch between tool size and seal size can create procedural problems, such as damage to the seal or insufflation gas leaks. Temporary loss of pressure during certain portions of the procedure is not catastrophic, as more insufflation gas can be delivered to reestablish insufflation, but preservation of insufflation is generally needed during the manipulation of tools as the procedure is carried out.

When a tool change is needed, an entire cannula seal can be exchanged, but switching out a cannula seal to accommodate a tool change during a procedure can be time consuming and inconvenient.

To avoid the need to change the cannula or cannula seal during a procedure, a seal component can be configured to accommodate a range of instrument shaft sizes. A seal that allows for the exchange of surgical instruments, without changing the seal itself, can avoid procedural difficulties and time-consuming extra steps during the procedure. A cannula seal with an expandable seal opening can accommodate a range of instrument shaft sizes and enable a tool exchange during a procedure without exchanging the cannula seal. However, in an expandable seal that depends on stretching of material to accommodate a shaft that is larger than a neutral size of a seal opening, friction can become a problem, especially when an instrument is significantly larger than the opening. When an instrument that is larger than a seal opening is inserted through the opening, normal forces between the seal wall and the instrument shaft can generate significant frictional forces, due to the resistance by the seal material to the stretching required to accommodate the instrument shaft. The friction forces can in some instances make it excessively difficult to move an instrument shaft or detect forces present at an instrument-tissue interface at the surgical site. Forces can also be a problem when a user is attempting to move an instrument shaft away from a neutral axis. When an instrument shaft that is moved away from a neutral axis, the seal tends to resist the off-axis movement and bias the instrument back toward the neutral axis. The friction forces created by the seal-instrument interface can also complicate the use of a force-sensitive control system. Some tele-robotically-assisted surgical systems, such as the Intuit Surgical da Vinci surgical system, can use force sensors to detect and communicate forces in the system. This force-sensitive capability can be particularly useful, for example, to convey to a user (e.g., a surgeon) the amount of force that is experienced between a surgical tool and tissue at the surgical site. Excessive frictional forces between a seal and an instrument shaft can make it more difficult to ascertain or communicate the forces present at the surgical tool.

Another problem that can arise with a stretchable seal opening is the development of a gap between the seal and an instrument shaft when the instrument shaft is moved off-axis, i.e. laterally toward a side wall of the cannula. For example, a circular opening that is stretched to accommodate lateral movement of an instrument shaft can distort into a “cat-eye” shape, which can produce one or more gaps through which insufflation gas can escape.

To accommodate a range of instrument shaft sizes while avoiding leakage, avoiding the development of undesired frictional or other forces between a seal and an instrument shaft, and preferably avoiding both leakage and undesired forces, a seal can be formed from a plurality of seal wall layers that have overlapping openings. The seal openings can be arranged so that they overlap, but do not coincide. In this arrangement, where seal openings overlap but do not coincide, a portion of one or more of the openings is covered or blocked by one or more layers. A region where the openings overlap but are not blocked by any layer can be considered an “effective opening” or “effective seal opening”.

An instrument shaft can be inserted through the effective opening to access a surgical site. To avoid or reduce leakage, it can be desirable to form an effective opening that has a size and shape that is slightly smaller than a minimum instrument shaft cross section. When an instrument shaft that is larger than that effective opening, or has a different shape than the effective opening, is inserted through the opening, one or more of the layers will be forced to move to accommodate the instrument shaft. When the actual opening in a particular layer is larger than the instrument shaft, that particular layer can accommodate the shaft by distorting the shape of the opening, e.g. distort a circle into an oblong shape, or bias a side of an ellipse outward away from the major or minor axis, or both axes. Changing the shape of an opening can require less stretching of seal material—and thus less force—than enlarging an opening that is smaller than the instrument shaft. By stacking a plurality of layers that each have relatively large openings, one can form an effective opening of a desired size that is smaller than the size of some or all of the individual openings, and allow the effective opening to be enlarged by an instrument shaft by deforming the shape of the individual openings in the seal layers. This form of expansion of the effective opening can reduce or avoid stretching of the seal material to accommodate an instrument shaft that is larger than the effective opening, and can thus reduce normal and frictional forces on the instrument shaft.

A multi-layer seal with overlapping openings that create an effective seal opening can also provide desirable performance characteristics when an instrument shaft is moved off-axis, e.g., away from an axis that can be perpendicular to the seal and extends through the effective opening. When the instrument shaft is biased away from the axis, one or more of the openings in the layers can be deformed to accommodate the movement. As the instrument is moved around relative to the axis, some of the seal layers can deform to accommodate the movement, while other relax toward a neutral state. In some examples, the effective opening can be thought of a “floating hole”, because the size and shape of the opening can be approximately constant while the center of the effective opening moves relative to the axis, due to the simultaneous deformation and relaxing (i.e. returning toward a neutral state) of the layers that form the openings. The movement of the seal layers that form the effective opening can reduce or eliminate distortion of the effective seal opening, which can avoid or reduce leakage of insufflation gas when the instrument shaft is moved off axis. For example, in some configurations, a “cat-eye” problem where deformation of a seal by off-axis movement of an instrument shaft creates a pathway for escape of insufflation gas—can be avoided by the simultaneous movement of the seal layers.

The effective opening can, for example, be a circle, or approximate a circle, to match the most common instrument shaft form factor—a cylinder. In an example, a plurality of layers can each have a circular opening, and the circular openings can overlap, but be offset from one another, i.e. non-concentric. The overlapping circular openings can be equally spaced to form a shape formed from a number of arcs of circles. Such a shape formed from arcs of circles can roughly approximate a circle, and can be distorted into a circle by an instrument shaft.

In an example, a plurality of circular openings each have a diameter that is at least twice the diameter of the minimum shaft diameter for which the seal is designed, i.e. at least twice the diameter of the smallest circle that can be drawn around the outside of the effective opening without intersecting the opening, to avoid leaving a gap between the instrument shaft and the seal, through which insufflation gas could escape. In some examples, each of the circular openings have a diameter that is at least three times the minimum shaft diameter. In some examples, each of the openings can have a diameter that is at least as large as the maximum instrument shaft for which the instrument is designed. In some examples, a portion of each opening is a circular or other type of arc, and the remainder of the opening is oblong or irregular.

In other examples, other non-circular shaped openings can be used to form a desired effective opening profile. For example, layers can be formed with oval or ellipse shaped opening. Ovular or elliptical openings can be advantageous because the ovular or elliptical shape can deform more, and stretch less, resulting in lower forces on the instrument shaft. Shapes can also be formed using parabola or hyperbola geometries that, as described above, allow an opening in a layer to deform to reduce or avoid stretching the seal material.

Other opening shapes are possible, and can be distorted to accommodate an instrument as described above. For example, an opening in a layer can be a rectangle, pentagon, octagon or other polygon, or an irregular angular or curved shape, which can include an arc of a circle, ellipse, parabola, or hyperbola.

The openings can all be the same size and shape, or can vary in size, vary shape, or both size and shape. In some examples, the layers include openings that are different sizes of the same shape, e.g. different sized circles, ellipses, or ovals. In some examples, the openings are not evenly spaced on the seal wall. Varying the size, shape, and configuration of the openings can allow for design of a seal with a desired performance or preferential direction or range of movement. For example, the openings can be designed to more easily accommodate movement along a selected axis, or in a particular direction or directions.

In some examples, an effective opening profile can be a Reuleaux polygon—a shape formed from a number of arcs of circles each having a center that is on one of the other arcs. For example, a three-layer seal component can be configured with openings that form an effective opening that has a Reuleaux triangle profile. Such a shape can be formed by three layers having circular openings. In another example, a four-layer seal component can be configured with openings that form a four-sided Reuleaux polygon. Such a shape can be formed by four layers each having a circular opening. Five-sided and six-sided Reuleaux polygons are also possible. Effective openings with a Reuleaux polygon profile can also be formed using layers that have openings that have circular arc features but do not form full circles.

A multi-layer seal can be provided as a seal assembly, for example by stacking or nesting a number of seal components that each have a seal opening. Other configurations are also possible. For example, layers can be situated on top of each other and secured in a desired configuration using mechanical techniques, such as clamping or other types of compressing layers together, sewing, mechanical bonding, locking features such as key, or a combination thereof. Layers can also be secured together using other techniques, such as chemical bonding (e.g., application of adhesive), along or in combination with mechanical techniques. In some examples, nesting sealing components can be provided with matching inner and outer tapers, which allows the components to fit together with little or no gaps between the components.

An expanding multi-layer seal assembly can be used with a surgical system that includes tools that allow a physician to see and manipulate tissue (or other objects or materials) inside a patient's body, using controls situated outside the patient's body. Visualization tools can, for example, include optical tools, such as fiber optic cameras, or electronic tools, such as digital cameras or sensors. Surgical tools can include, for example mechanical or electromechanical tools such as needle drivers, suture tools, retraction instruments, clip appliers, probes, fenestrated graspers, or cardiac stabilizers. Surgical tools can also include energy instruments such as monopolar or bipolar tools, ultrasonic tools, or lasers, which can be used for cautery or ablation, for example. Tools can be coupled to a computer system and electromechanical manipulators to provide precision and ease of use for a physician or clinical personnel. The use of such systems is sometimes referred to as a robot-assisted minimally invasive surgery.

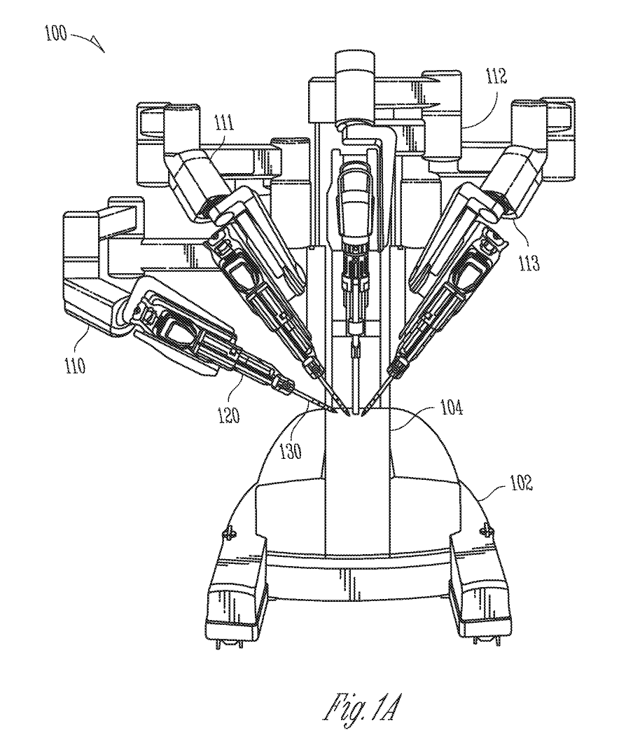

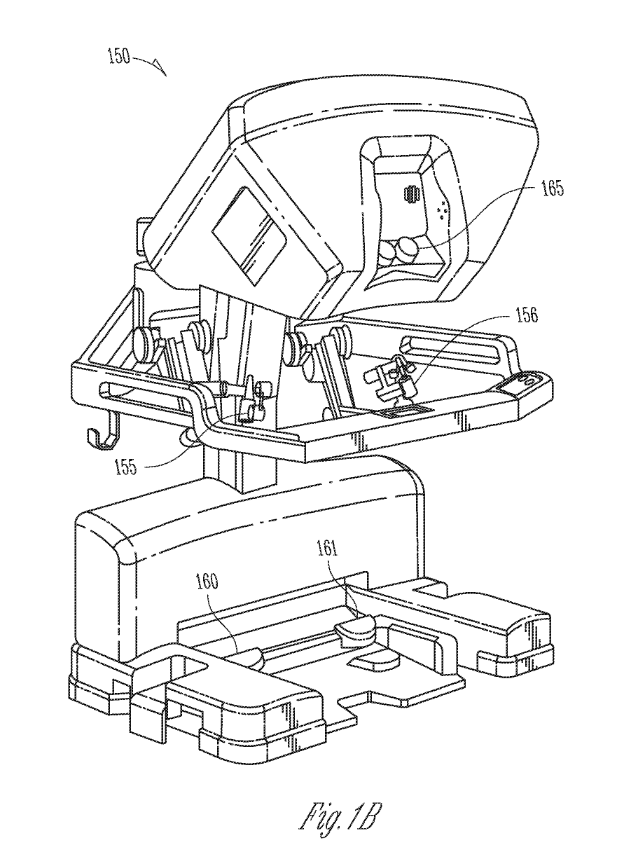

FIGS. 1A, 1B, and 1C illustrate an example robot-assisted minimally invasive surgical system. FIG. 1A shows an instrument system 100 (sometimes known as a “patient side cart”) that can be situated near a patient operating table (not shown). FIG. 1B shows a surgeon console 150 that can include controls and a viewing system. FIG. 1C shows a control cart 175 that can include, for example, processing equipment and communication equipment.

Referring again to FIG. 1A, the system 100 can include a base 102, a support tower 104, and one or more manipulator arms 110, 111, 112, 113, which can be mounted on the support tower. Alternatively, the manipulator arms 110, 111, 112, 113 can be connected to a main boom (not shown), which can be movable. An instrument 130 can be mounted to an instrument mount 120 on one of the manipulator arms. A cannula (not shown in FIG. 1A) can be mounted to a cannula mount. An instrument 130 can be inserted through a cannula seal in the cannula, and into the patient (not shown) for use in a surgical or other medical procedure. Through movement of the manipulator arms, the orientation of the instrument can be controlled in multiple dimensions, e.g. lateral, horizontal, vertical, angular movements in one, two, or three planes.

FIG. 1B shows an example physician console 150. The physician console can include hand control 155, 156 and pedal controls 160, 161. The hand controls 155, 156, and pedal controls 160, 161 can be used to control equipment at the patient side cart. For example, portions of a distal end of an instrument can be manipulated using instrument controls. The controls can include haptic feedback features so that a physician can interpret physical information, such as resistance or vibration, through the controls. The physician console 150 can also include a viewing system 165 that can display video or other images of a surgical site.

FIG. 1C shows an example control cart 175. The control cart can include processing equipment 180 for processing controls, facilitating communication between the physician console and the patient side cart, or a remote site. The control cart 175 can also include a display 190, which can show images that the physician is seeing on the physician console, a video feed from a camera in the patient, or other information. In an example configuration, signals input at a surgeon console 150 can be transmitted to the equipment 180 on the control cart, which can interpret the inputs and generate commands that are transmitted to the patient side cart 100 to cause manipulation of an instrument 130 or portions of a manipulator arm 110. The equipment 180 is shown on a cart for exemplary purposes, but could also be arranged in various configurations, e.g., it could be integrated as part of the physician console, the patient side cart, or both, or divided between the physician console and patient side cart. The equipment can also be provided as software, hardware, or both, on an installed or remote system.

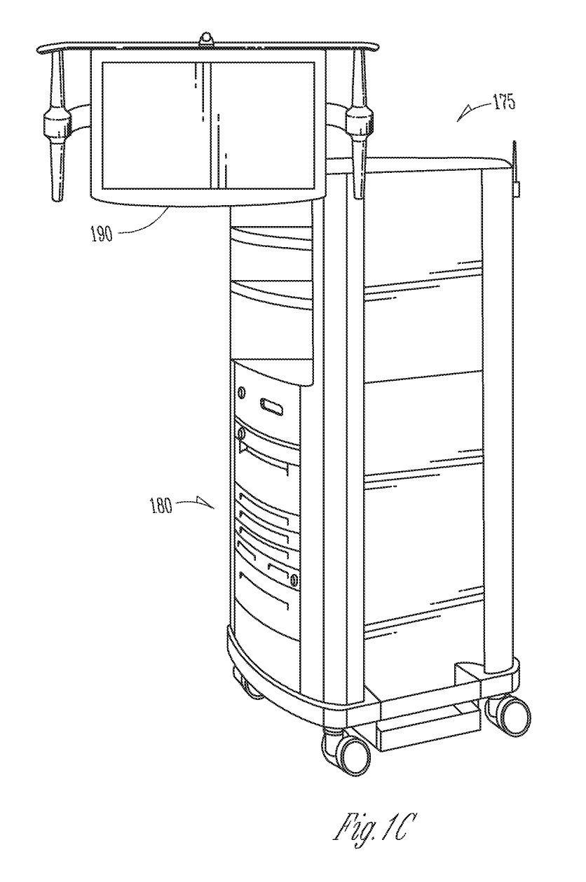

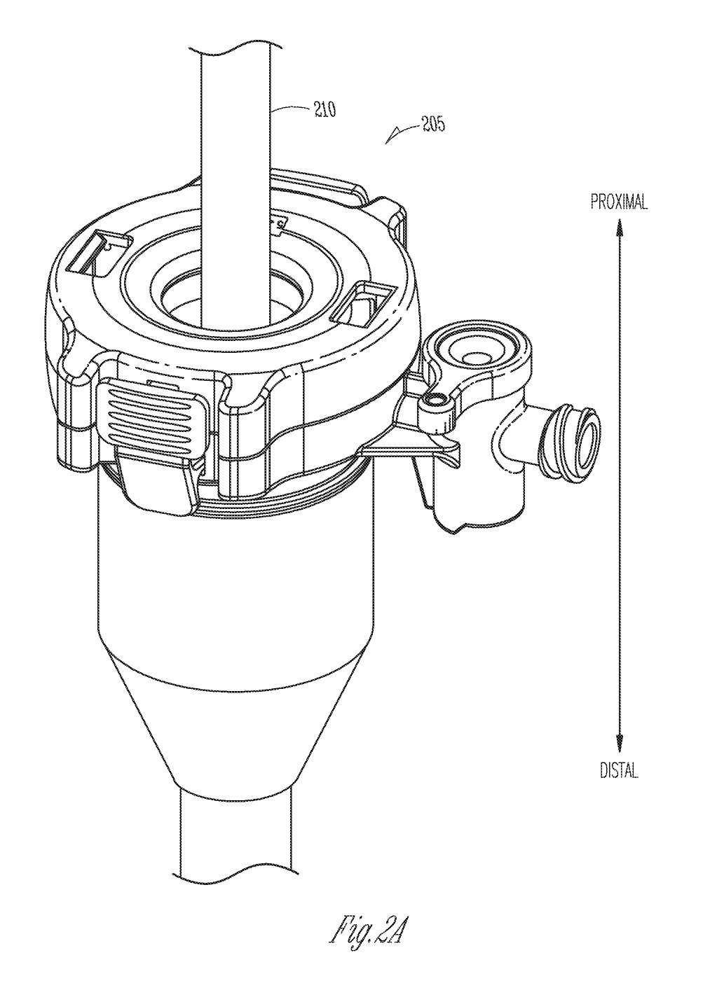

FIG. 2A and FIG. 2B show an example of an expandable cannula seal within a cannula 205 in a minimally invasive surgery system. An instrument shaft 210 can be inserted into through a cannula seal assembly, which includes an expandable cannula seal. The cannula seal can prevent or reduce leakage around the instrument shaft 210.

FIG. 2B shows the cannula 205 utilized in a single site surgical approach, where multiple instrument shafts can be inserted through cannulas 215, 220 that are inserted through a single incision site in the skin 230, such as through the navel. A tool such as an end effector or a visualization device can be situated on a distal end of an instrument shaft 210 that is inserted through an expandable cannula seal. An end effector utilized in the surgical system can be a jawed surgical end effector, such as a scissors, grasping retractor, or needle driver, for example. A visualization device can be a digital video camera or endoscope, for example. An expandable cannula seal can allow for exchange of instruments during the procedure without changing the cannula seal, even if the instruments require or include shafts of varying dimensions, because the cannula seal can expand to accommodate different shaft sizes.

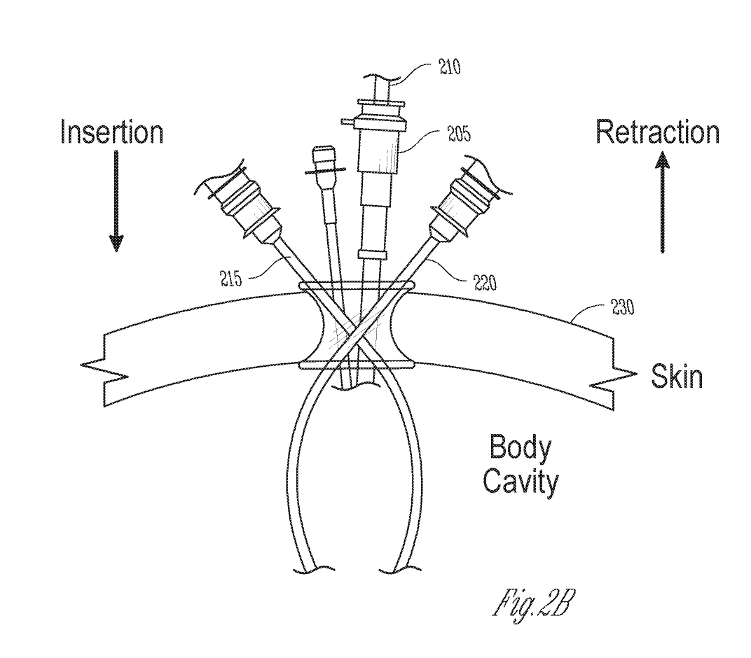

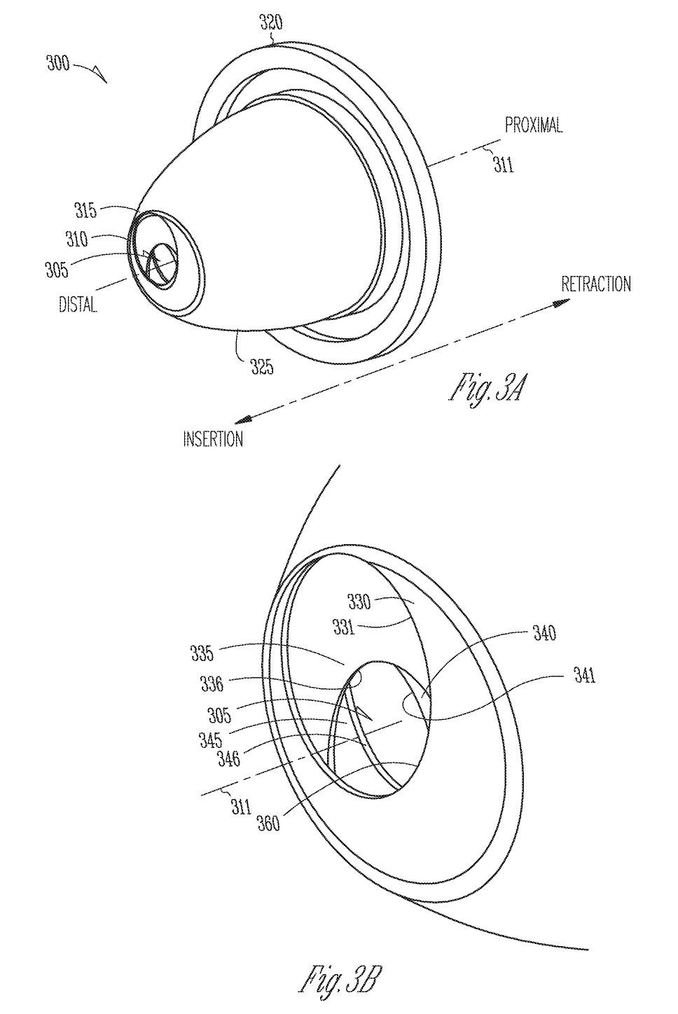

FIG. 3A is a perspective illustration of an example seal component 300. The seal component 300 can include a distal seal wall 310 at a distal end 315 of the seal component, a proximal end 320, and a tapered portion 325 that extends between the distal end 315 and the proximal end 320. The distal seal wall 310 can include seal layers that have overlapping openings that form an effective opening 305 that can be aligned with a seal component axis 311. The openings can be offset from the seal component axis 311. An instrument can be inserted along the seal component axis.

FIG. 3B is an enlarged view of a portion of the seal component 300 of FIG. 3A, showing the overlapping openings in the seal layers. Layer 330 includes an opening 331. Layer 335 includes opening 336. Layer 340 includes opening 341. And layer 345 includes opening 346. The openings 331, 336, 341, 346 together form the effective opening 305. The edges of the layers 330, 335, 340, 345 define the profile 360 of the opening 305.

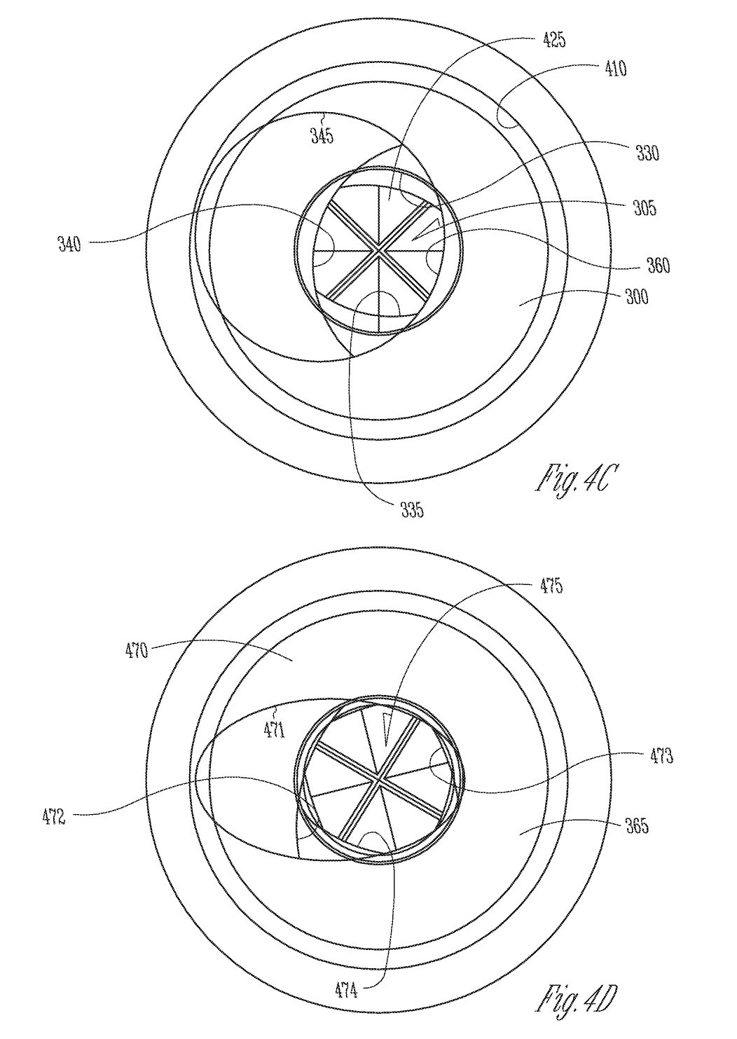

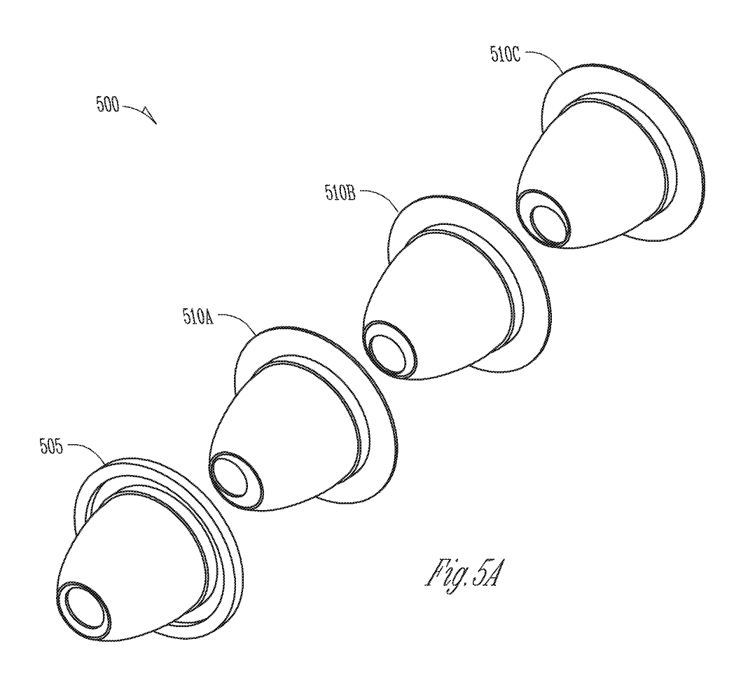

An instrument shaft can be inserted through the effective opening 305. The layers 330, 335, 340, 345 will deform to change the shape of the openings 331, 336, 341, 346 to accommodate the instrument shaft. In an example, the layers can be formed by assembling nesting components, as previously described, and as shown in FIG. 5A. The layers can also be adhered together, compressed, mechanically or chemically attached, or otherwise formed to work together to seal against an instrument shaft.

In an example, the seal component 300 can be formed of polyisoprene, which can provide good flexibility and tear resistance. In other examples, the seal component 300 can be formed of silicone or other rubber materials.

FIG. 3C is a top plan view of the seal component 300 of FIGS. 3A and 3B. The hidden portion of the overlapping openings are shown as dashed lines. The seal axis 311 can be perpendicular to and centered in the distal seal wall 310.

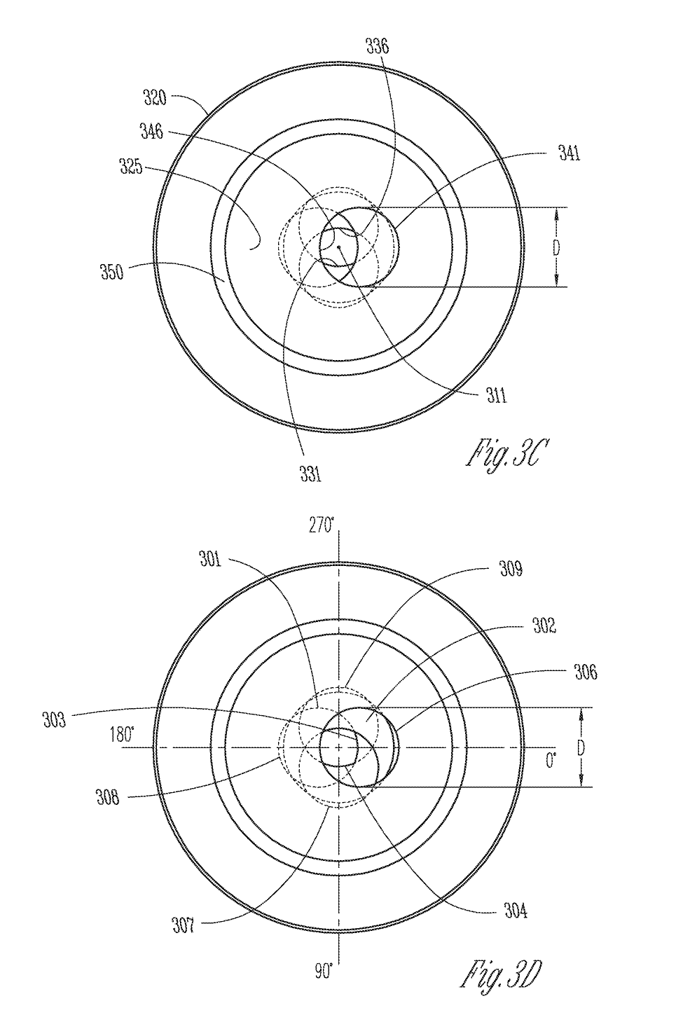

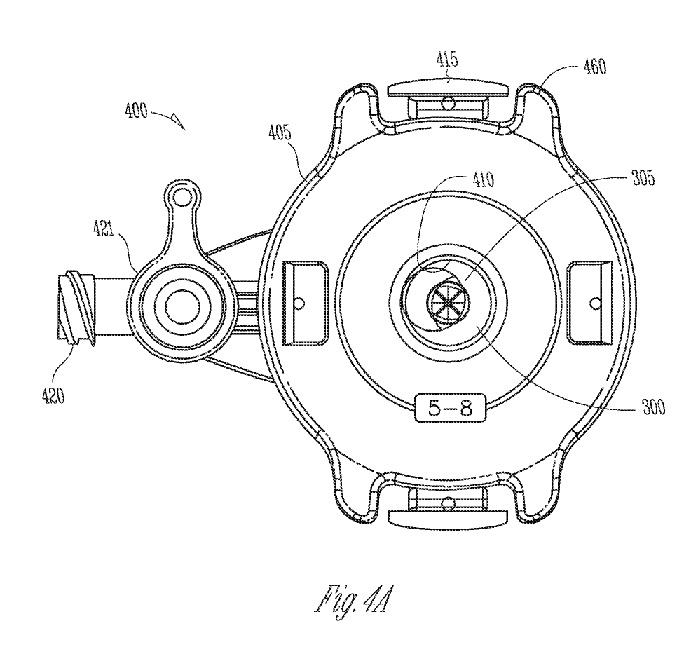

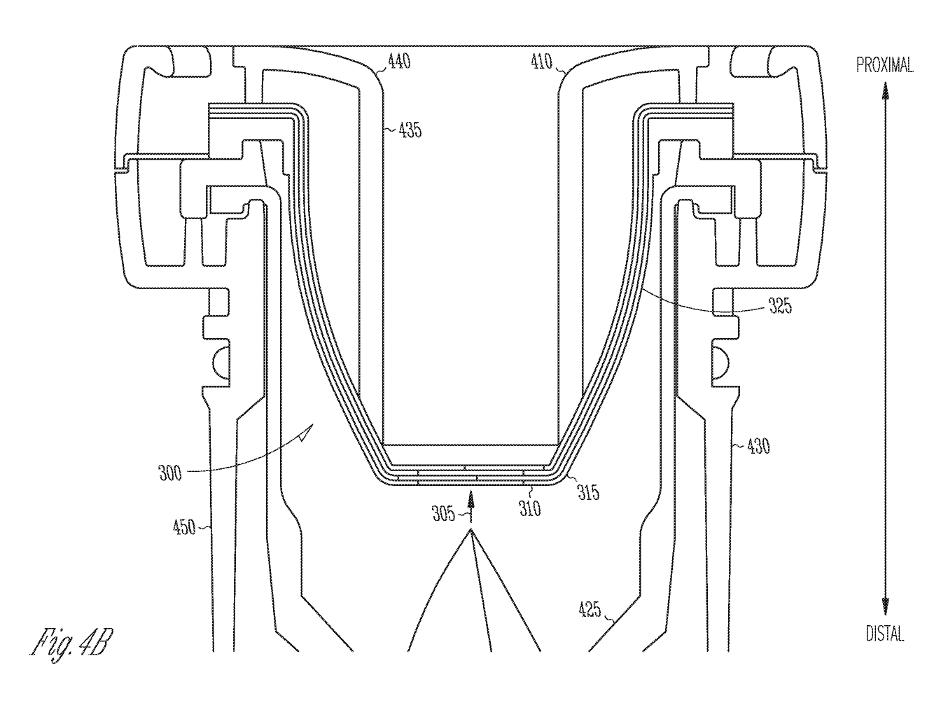

In the example shown in FIG. 3C, the openings 331, 336, 341, 346 are circular, and the circular openings are evenly spaced about the seal axis 311. As shown in FIG. 3C, the openings can be arranged so that top layer 340 has an opening 341 that is horizontally offset from opening 346 in the next layer 345. Below layer 345, opening 336 on layer 335 is vertically offset from opening 331 on layer 330. This configuration can also be seen in FIG. 4C.

FIG. 3D is a top plan view of another example seal component showing an alternate arrangement of overlapping openings. In this example, the seal is configured so that the placement of the openings is in a clockwise fashion when considered from the top layer to the bottom layer: Circular opening 306 is in the top layer and offset from the center of the seal at zero degrees (i.e. offset to the right as viewed in the top plan view), opening 307 is in the layer below the top layer and offset from the center of the seal at 90 degrees (i.e. vertically offset downward in the top plan view), opening 308 is in the next layer and offset from the center of the seal at 180 degrees (i.e., offset to the left in the top plan view), and the opening 309 is in the bottom layer and offset from the center of the seal at 270 degrees (i.e., vertically offset upward in the top plan view.) Various other options are also possible. For example, the openings could be placed to proceed in a counter-clockwise order from top to bottom (i.e. 0 degrees, 270 degrees, 180 degrees, 90 degrees) or any possible alternating order (e.g., 0 degrees, 180 degrees, 90 degrees, 270 degrees.)

FIG. 4A is a top view of a seal assembly 400 incorporating the seal component of FIG. 3A. The seal assembly 400 can include a cap 405 that has an opening 410 through which seal component 300 and the effective opening 305 can be accessed. Insufflation gas can be delivered through port 420 and controlled with valve 421.

FIG. 4B is a side cross-sectional view of the seal assembly of FIG. 4A. An instrument and instrument shaft (not shown) can be inserted at a proximal end of the opening 410 in the cap. A guide portion 435 can be configured to guide an inserted instrument toward the effective opening 305 in the distal seal wall 310 of the distal end 315 of the seal component 300. The seal component 300 can be secured between the cap 405 and a lower portion 450 of the seal assembly 400.

FIG. 4C is an enlarged view of a portion of the seal assembly 400 of FIG. 4A that shows the layers 330, 335, 340, 345 that define the profile 360 of the effective opening 305 of the seal component 300. In FIG. 4C, an additional seal 425, which can be a cross-slit seal, for example, is visible through the effective opening 305.

While the openings are illustrated as circles, the openings in the seal layers can be selected from a variety of possible shapes, such as ellipses, ovals, oblong shapes, Reuleaux polygons, or other shapes. FIG. 4D is a top view of a portion of a seal assembly that has an example seal component 365 that has elliptical openings. A top layer 470 has an elliptical opening 471. Additional layers 472, 473, 474 can each have an elliptical opening similar to opening 471. The layers and openings can be configured to create an effective opening 475 that can closely approximate a circle.

A seal component that has multiple seal layers can be constructed as an assembly of seal components that each have a seal opening. FIG. 5A is an exploded assembly view of four seal sub-components 505, 510a, 510b, 510c, that can be assembled into a seal component that has multiple seal layers. In an example configuration, seal component 505 can be a first type of component, which optionally can include structural features to facilitate assembly with other seal components, or assembly into an assembly such as the assembly 400 shown in FIGS. 4A and 4B. Seal sub-components 510a, 510b, and 510c can be a second type of components, and can be similar, or identical, to each other. In various examples, additional or fewer components or types of components can be used, which can result in additional or fewer layers. A component similar to seal sub-components 510a, 510b, and 510c can optionally be used in lieu of seal component 505.

FIG. 5B is a perspective view of a first type of seal sub-component 505. FIG. 5B is a perspective view of a second type of seal sub-component 510. The first type of sub-component 505 includes a structural ring 515 that can, for example, but used to couple to other parts of a seal assembly, such as a cap, or a cannula or other lower part of a seal assembly. The structural ring 515 can also optionally interface with a lip 525 on the second type of sub-component 510 to facilitate assembly. In an example, the under-side 535 of the lip 525 on the second type of sub-component can fit against the top side of the structural ring 515. In an example, the sub-component 505 can include notches on the structural ring 515 or elsewhere that are sized and shaped to interface with key features on subcomponent 510. In another example, the sub-component 505 can include key features that interface with notches or additional key features on the second type of sub-component 510.

As shown in FIG. 5A, the second type of sub-component can be sized and shaped to enable nesting of sub-components 505, 510a, 510b, 510c in an assembly. For example, the inner and outer tapers of components 505, 510a, 510b, 510c can be the same or similar to allow for a close fit between the components. The wall thickness of the tapered portions 540, 545 and the end walls 550, 555 of the seal sub-components 505, 510a, 510b, 510c can also be similar or the same to provide a close fit or consistent performance. In some examples, the stacking of nested components can provide desirable strength characteristics or puncture resistance, due to the multiple layers of materials in the tapered portions 540, 545 and the end walls 550, 555.

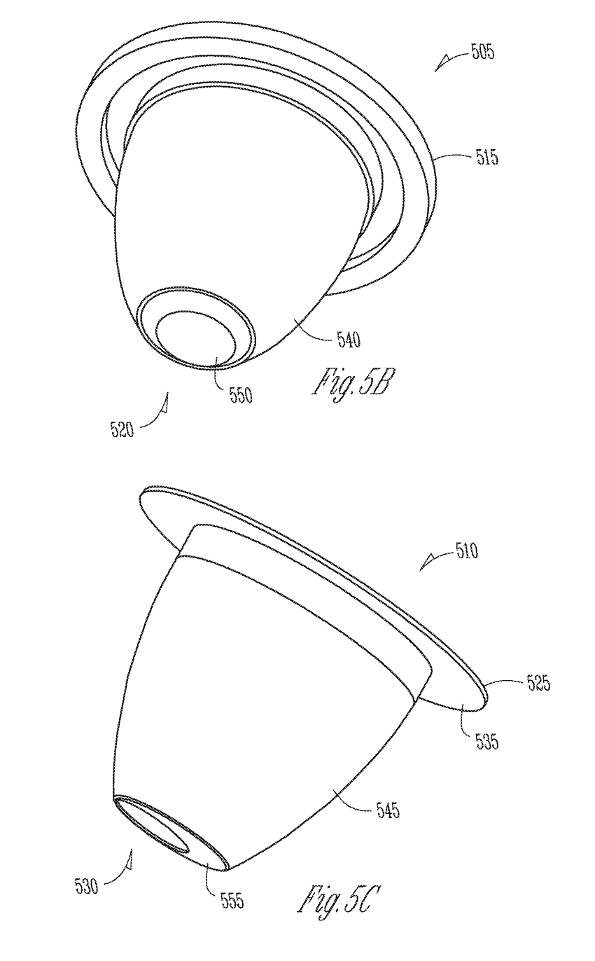

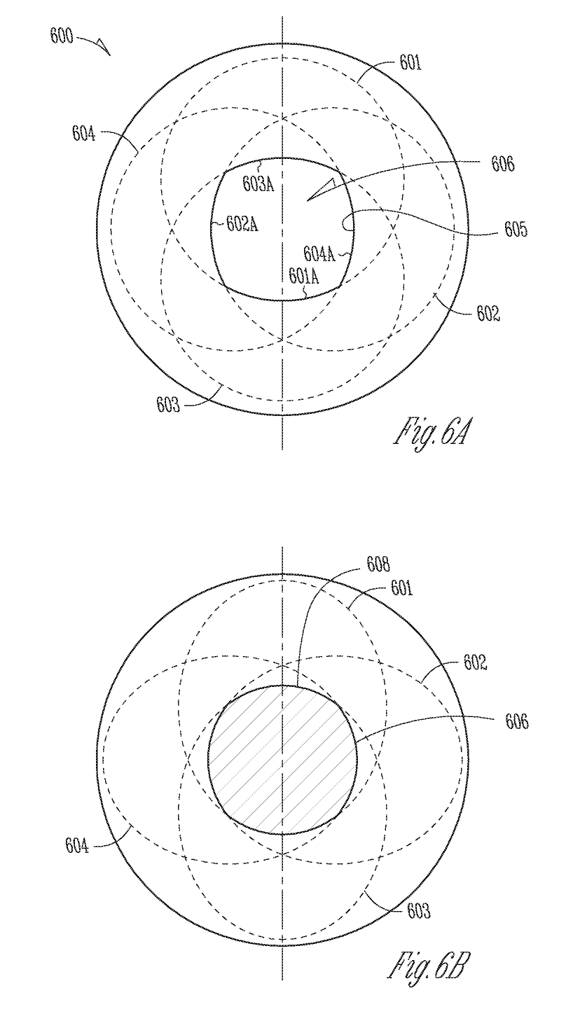

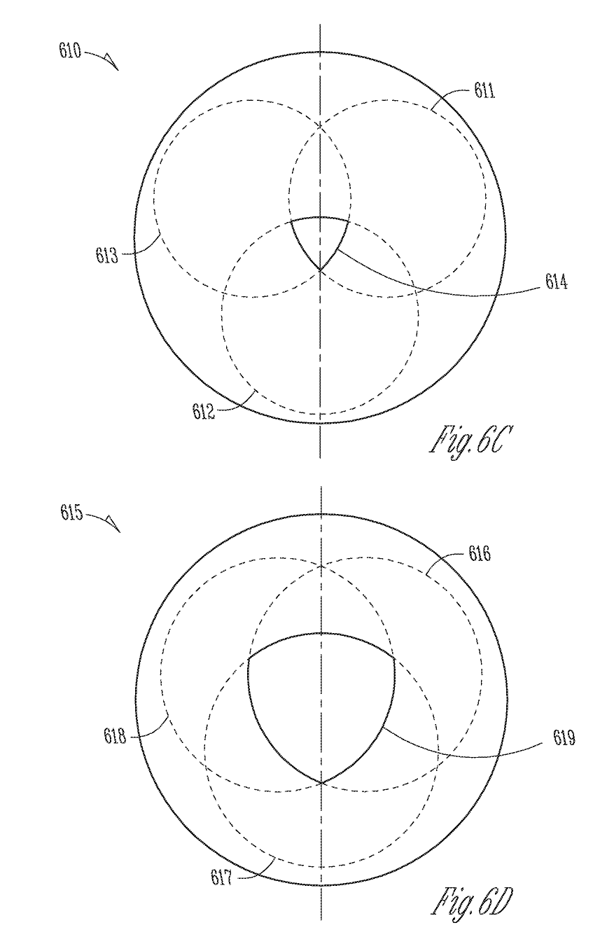

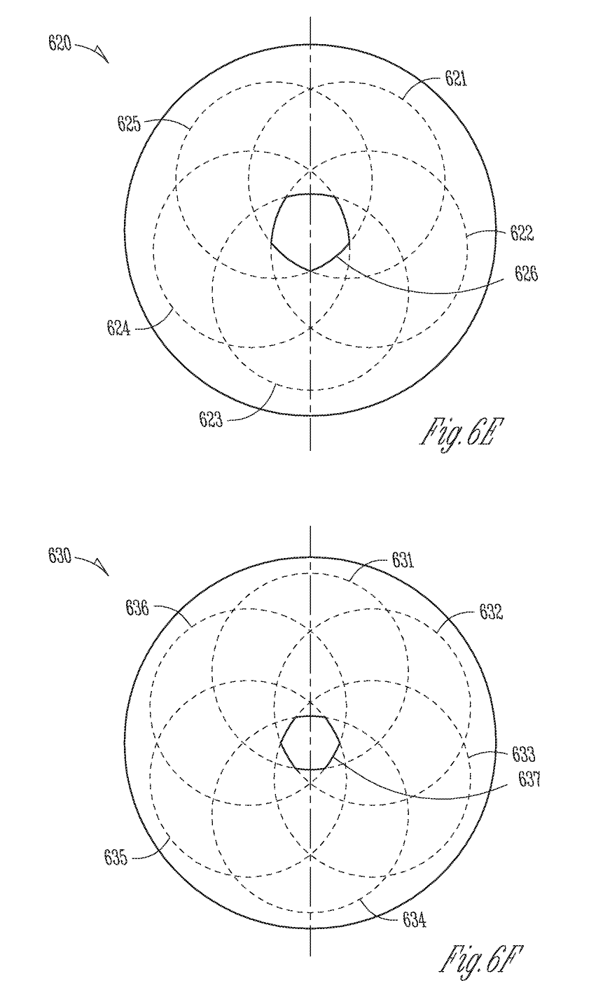

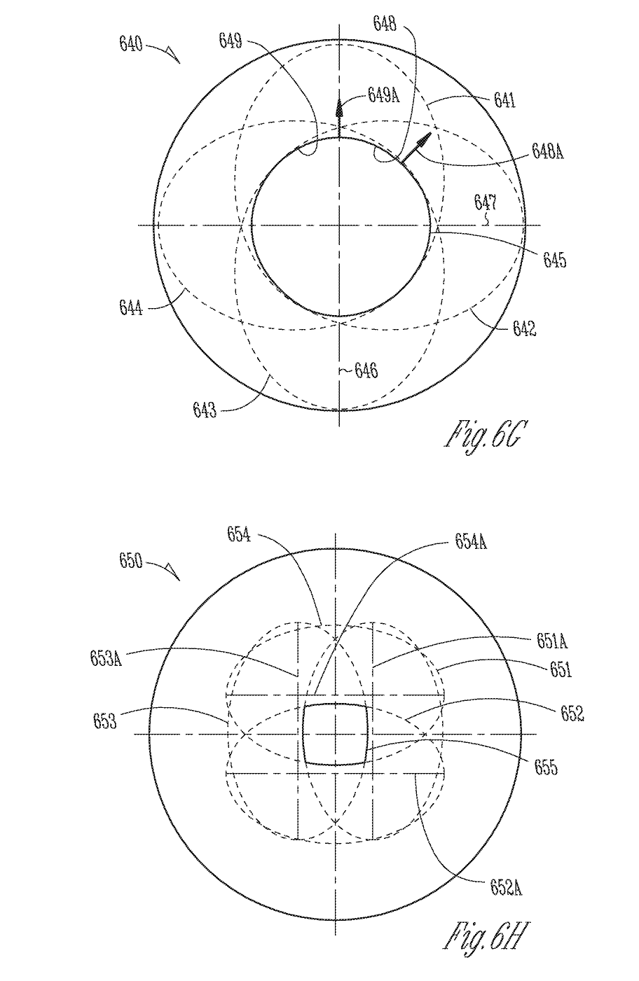

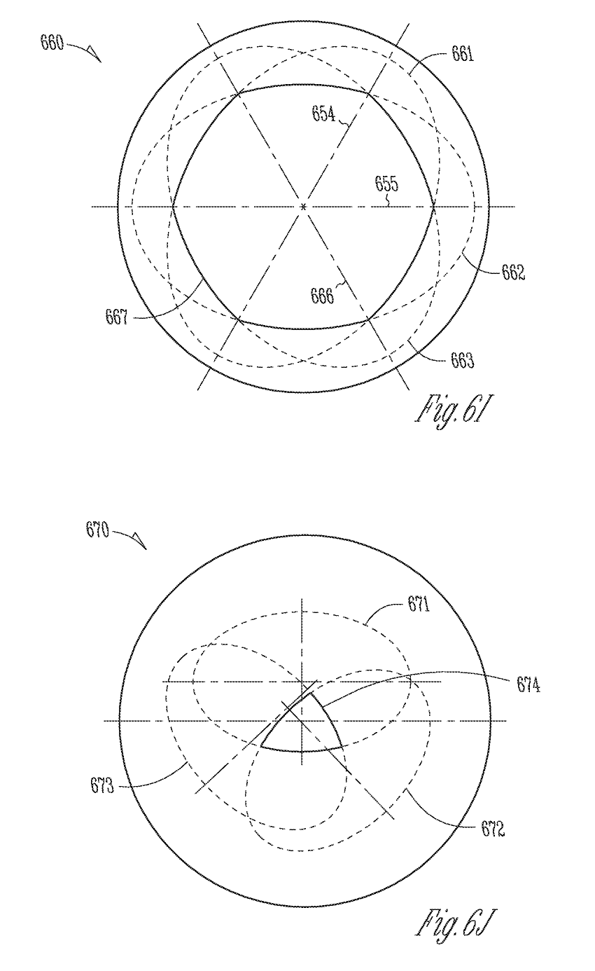

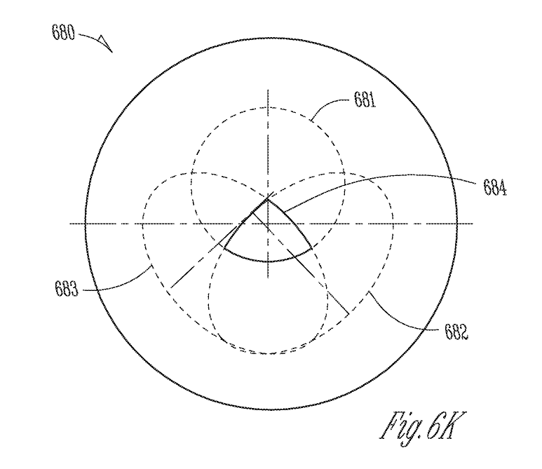

FIGS. 6A to 6J are top plan view illustrations of a variety of seal component configurations having different size, location, and shape openings.

FIG. 6A is a top plan view of a portion of a seal component 600 illustrating an example arrangement of four circular openings 601, 602, 603, 604. Portions 601a, 602a, 603a, 604a of the edges of the seal layers define a profile 605 of the seal effective opening 606 through which an instrument can be inserted. In an example where the centers of the circular openings 601, 602, 603, 604 are on one of the other circles, the effective opening 606 is a four-sided Reuleaux polygon (i.e. Reuleaux square).

In an example, a seal has a plurality of layers that each has a circular opening having a diameter of at least 8 millimeters, and the layers overlap to form an effective opening of 3.8 mm to 4.4 mm (preferably 4.1 mm), to accommodate a range of instrument shaft sizes from 5 to 8 millimeters.

FIG. 6B is a top plan view of the portion of the seal component of FIG. 6A illustrating a cross-section of a shaft 608 inserted through the effective opening 606. The effective opening 606 has been expanded to accommodate the circular cross-section of the shaft 608. To accommodate the shaft 608, the circular openings have been distorted into elliptical-shaped openings.

FIG. 6C is a top plan view of a portion of a seal component 610 illustrating an example arrangement of three circular openings 611, 612, 613 that form an effective opening 614 that has three curved sides.

FIG. 6D is a top plan view of a portion of a seal component 615 illustrating another example arrangement of three circular openings 616, 617, 618 that form an effective opening 619. The three circular openings shown in FIG. 6D are larger and further apart than the openings shown in FIG. 6C, resulting in a larger effective opening 619 that again has three curved sides.

FIG. 6E is a top plan view of a portion of a seal component 620 illustrating an example arrangement of five circular openings 621, 622, 623, 624, 625 that define an effective seal opening 626 that has five curved sides.

FIG. 6F is a top plan view of a portion of a seal component 630 illustrating an example arrangement of six circular openings 631, 632, 633, 634, 635, 636 that define an effective seal opening 637 that has six curved sides.

FIG. 6G is a top plan view of a portion of a seal component 640 illustrating an example arrangement of four elliptical openings 641, 642, 643, 644 that define an effective seal opening 645 that can closely approximate a circle. In this example, ellipse 641 and ellipse 643 share a major axis 646, and ellipse 642 and 644 share a major axis 647.

FIG. 6H is a top plan view of a portion of a seal component 650 illustrating another example arrangement of four elliptical openings 651, 652, 653, 654 that define an effective seal opening 655 that has four curved sides. In this example, the major axis 651a of ellipse 651 is parallel to but offset from the major axis 653a of ellipse 653, and the major axis 652a of ellipse 652 is parallel to but offset from the major axis 654a of ellipse 654. In an example, the ellipses can be asymmetrically arranged to provide a desired bias toward a side of the seal. In other examples, the ellipses can be evenly or symmetrically arranged to form an opening that has a center that is concentric with the center of the seal.

FIG. 6I is a top plan view of a portion of a seal component 660 illustrating an example arrangement of three elliptical openings 661, 662, 663. In some examples, three elliptical openings are concentric—i.e. they have the same center (the intersection of the major and minor axis of an ellipse). The major axes of the ellipse can be angularly displaced, as shown in FIG. 6I. In an example, the three elliptical openings 661, 662, 663 can have respective major axes 664, 665, 666 that are each 60 degrees from the closest major axis, which results in an even distribution. In other words, axis 664 and axis 665 form a 60 degree angle, and axis 665 and axis 666 form a 60 degree angle. The three elliptical openings can overlap to form an effective opening 667 that has six curved sides. In some examples, the elliptical openings can be offset from one another (i.e. non-concentric). In some example, the openings 661, 662, 663 can have an oblong or irregular shape, and such openings can be symmetric, concentric, or offset.

FIG. 6J is a top plan view of a portion of a seal component 670 illustrating another example arrangement of three elliptical openings 671, 672, 673 that form an effective opening 674. In an example, the minor axes are each one hundred and twenty (120) degrees from both of the other minor axes, but the ellipses are off-set, e.g. non-concentric. The elliptical openings can define an effective opening that has three curved sides. In other examples, the off-set ellipses can form an elongated shape, which can better accommodate a non-round instrument. Some camera tools, for example, have an oblong or other non-round cross section. In some examples, the effective opening 674 can also accommodate a round (e.g., circular) instrument. In some examples, the openings can be configured as described above, but can have an ovular, oblong, or irregular shape.

FIG. 6K is a top plan view of a portion of a seal component 680 in which one opening 681 is a circle and two openings 682, 683 are ellipses. Other layer combinations with varying opening shapes or sizes are possible, such as one circular opening and two elliptical openings, two elliptical openings and two circular openings, or a plurality of elliptical or other shaped openings in varying shapes or sizes.

In the various configurations shown in FIGS. 3A through 6K, it is typically desirable that the geometries of the openings and the effective opening be symmetrical to avoid excessive variability in force or performance characteristics at points around the effective opening. This means that the sides of curved-polygon effective openings will typically be the same (equilateral). The force characteristics will vary, however based upon the direction in which an instrument shaft is biased against the seal. For example, in FIG. 6G, at point 648 an instrument shaft will need to move two layers of material to move perpendicular to the effective opening in direction 648 A, whereas at point 649 the shaft will need to move only one layer of material to move perpendicular to the effective opening in direction 649. To reduce this variation, it may be desirable to use fewer layers, e.g. three or four layers instead of five or six, to reduce the percentage of plausible shaft displacements in which two or more layers must be moved to accommodate the shaft. For similar reasons, it can also be desirable to limit the number of intersections of the openings, e.g. a configuration of ellipses arranged as shown in FIG. 6G or 6J can be preferable to the configuration shown in FIG. 6I. Limiting the number of layers can also limit the amount of frictional forces imposed on the shaft: In an example, three to five layers is preferred to avoid excessive friction.

In the various configurations shown in FIGS. 3A through 6K, it can be desirable to form relatively large openings in the seal layers, as larger openings can correlate to lower forces and less friction on an inserted instrument shaft. In an example, a plurality of circular openings each have a diameter that is at least twice the diameter of the minimum shaft diameter for which the seal is designed, i.e. the smallest circle that can be drawn around the inside of the effective opening without intersecting the opening. In some examples, the circular openings have a diameter that is at least three times the minimum shaft diameter. In various examples, the circular openings are at least as large as the maximum diameter instrument shaft for which the seal is designed to avoid stretching an opening to accommodate an instrument shaft that is larger than the opening.

In the various configurations shown in FIGS. 3A through 6K, an effective opening of about 4.1 millimeters can be used to accommodate a shaft ranging in size from 5 millimeters up to as a large as 14 millimeters. Where larger sizes need to be accommodated, an effective opening of larger than 4.1 millimeters can be formed to accommodate to provide desirable sealing, friction force and deflection force characteristics. In any case, a “created circle” drawn from the outer boundaries of the effective opening should typically be smaller than the minimum instrument diameter for which the seal is designed, to assure that insufflation gas does not leak through opening between the instrument shaft and the instrument seal.

A seal component can also be designed to accommodate an instrument shaft that is not round (i.e. a non-cylindrical shaft). Some camera shafts, for example, have an oblong cross section. Openings in seal layers can be configured to define an oblong effective opening. In some examples, such an opening could work with larger circular shafts (large enough to fill the effective opening). In an example, an elliptical effective opening can be designed with a major axis that is similar in size to a cylindrical shaft, and a minor axis that is similar in size to a cross-dimension of an oblong shaft or a minor axis of an elliptical shaft.

Any of the example components described herein can be formed of a material that provides good flexibility and tear resistance, such as polyisoprene. Components can also be formed of silicone or other rubber materials.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A seal for sealing against a surgical instrument shaft, the seal comprising:
a first seal layer having a first seal opening; and
a second seal layer having a second seal opening, the second seal opening overlapping with the first seal opening, an effective seal opening being defined at least in part by the first seal layer and the second seal layer at a region where the first seal opening overlaps with the second seal opening, the effective seal opening being smaller than the first seal opening and the second seal opening, and the effective seal opening being sized and shaped to seal against an instrument shaft when an instrument shaft is inserted through the effective seal opening.

2. The seal of claim 1, wherein when an instrument shaft that is larger than the effective seal opening is inserted through the effective seal opening, the first seal opening or the second seal opening deforms to accommodate the instrument shaft.

3. The seal of claim 1, wherein the first seal opening has a first center, and the second seal opening has a second center, the second center being offset from the first center.

4. The seal of claim 3, further comprising a third seal layer having a third seal opening, the third seal opening having a third center that is offset from the first center and the second center.

5. The seal of claim 4, further comprising a fourth seal layer having a fourth seal opening, the fourth seal opening having a fourth center that is offset from the first center, the second center, and the third center.

6. The seal of claim 1, wherein the first seal opening and the second seal opening both have a circular shape.

7. The seal of claim 1, wherein the first seal opening has an elliptical, ovular, or oblong shape.

8. The seal of claim 1, wherein the first seal opening is a different size or different shape than the second seal opening.

9. The seal of claim 1, wherein the seal includes:
a first seal component that includes a first end wall that includes the first seal layer and the first seal opening, and a first side wall connected to the first end wall, the first side wall extending around and defining a first interior chamber, and
a second seal component that includes a second end wall that includes the second seal layer and the second seal opening, and a second side wall connected to the second end wall, the second side wall extending around and defining a second interior chamber, the second seal component sized and shaped to fit inside the first interior chamber of the first seal component.

10. The seal of claim 9, wherein the seal further includes a third seal component that includes a third end wall and a third side wall connected to the third end wall, the third side wall extending around a third interior chamber, the third seal component sized and shaped to fit inside the second interior chamber of the second seal component, the third end wall including a third seal layer and a third seal opening.

11. The seal of claim 10, wherein the seal further includes a fourth seal component that includes a fourth end wall and a fourth side wall connected to the fourth end wall, the fourth side wall extending around a fourth interior chamber, the fourth seal component sized and shaped to fit inside the third interior chamber of the third seal component, the fourth end wall including a fourth seal layer and a fourth seal opening in the fourth seal layer, the first seal layer, the second seal layer, the third seal layer, and the fourth seal layer defining the effective seal opening at a region where the first seal opening, the second seal opening, the third seal opening, and the fourth seal opening overlap.

12. A multi-layer instrument seal comprising:
a first seal component having a first seal wall and a first opening in the first seal wall, and a first side wall connected to and extending around the first seal wall, the first side wall defining a first interior chamber;
a second seal component having a second seal wall and a second opening in the second seal wall, and a second side wall connected to and extending around the second seal wall, the second side wall defining a second interior chamber, the second seal component being sized and shaped to fit inside the first interior chamber of the first seal component; and
a third seal component having a third seal wall and a third opening in the third seal wall, and a third side wall connected to and extending around the third seal wall, the third side wall defining a third interior chamber, the third seal component being sized and shaped to fit inside the second interior chamber of the second seal component;
wherein the first opening, the second opening, and third opening overlap with each other, an effective opening being defined by portions of first seal component, second seal component, and third seal component inside a region that intersects with the first opening, the second opening, and the third opening, the multi-layer instrument seal being sized and shaped to seal against an instrument shaft when the instrument shaft extends through first opening, the second opening, and the third opening.

13. The multi-layer instrument seal of claim 12, further comprising a fourth seal component having a fourth seal wall and a fourth opening in the fourth seal wall, and a fourth side wall connected to and extending around the fourth seal wall, the fourth side wall defining a fourth interior chamber, the fourth seal component being sized and shaped to fit inside the third interior chamber of the third seal component, the effective opening being defined by a region that intersects with the first opening, the second opening, the third opening, and the fourth opening.

14. The multi-layer instrument seal of claim 12, wherein the first opening, the second opening, and the third opening are equally spaced from each other.

15. The multi-layer instrument seal of claim 12, wherein the first opening, the second opening, and the third opening are the same size.

16. The multi-layer instrument seal of claim 12, wherein when an instrument shaft that has a cross-section that is larger than the effective opening is inserted through the effective opening, the seal applies normal forces against the instrument shaft that are smaller than the forces that would be created if the seal was a single layer of thickness and had an opening that is coextensive with the effective opening.

17. The multi-layer instrument seal of claim 12, wherein the multi-layer instrument seal has a proximal opening, the proximal opening and the effective opening defining an axis extending from the proximal opening to the effective opening, the instrument shaft being insertable through the proximal opening and the effective opening along the axis;
wherein in a first state the instrument shaft is in a first position aligned with the axis, and in a second state the instrument shaft is at a second position offset from the axis, in the second state one or more of the first seal wall, the second seal wall, and the third seal wall are stretched to deform one or more of the first opening, the second opening, or the third opening, wherein the portions of first seal component, second seal component, and third seal component that define the effective opening seal against the instrument shaft in both the first state and the second state.

18. The multi-layer instrument seal of claim 17, wherein in a third state the instrument shaft is offset from the axis to a third position that is different from the second position, and when instrument shaft is moved from the second position to the third position, one or more of the first seal wall, the second seal wall, and the third seal wall are stretched, and one or more of the first seal wall, the second seal wall, and the third seal wall are relaxed, so that the by portions of first seal component, second seal component, and third seal component that form the effective opening seal against the instrument shaft in the third state.

19. A method comprising:
receiving an instrument shaft along an axis through an effective opening in a multi-layer seal that seals against the instrument shaft, at least a portion of the effective opening formed by a first seal layer having a first opening and a second seal layer having a second opening, the second opening overlapping and not coextensive with the first opening; and
when the instrument shaft is moved to a first position off the axis, stretching one or more of the first seal layer and the second seal layer to move the effective opening toward the first position of the instrument shaft, and
when the instrument shaft is moved from the first position to a second position off the axis, relaxing the first seal layer and stretching the second seal layer to adjust the position of the effective opening to match the second position of the instrument shaft.

20. The method of claim 19, wherein the first seal layer has a first thickness, and wherein moving the instrument shaft off the axis a first distance creates less distortion in the effective opening than would occur in a comparable single-layer seal formed of a single layer having a comparable thickness that is equal to the first thickness and a comparable opening that is the same size and the effective opening.
